(19) United States
(12) Patent Application Publication
(10) Pub. No.: US 2021/0054438 A1
(43) Pub. Date: Feb. 25, 2021

(54) Methods for Determining Bound and Unbound Regions in Nucleic Acid Molecules and Systems for Practicing Same

(71) Applicant: The Regents of the University of California, Oakland, CA (US)

(72) Inventors: Angela Brooks, Santa Cruz, CA (US); Hinrich Boeger, Santa Cruz, CA (US); Eva Robinson, Santa Cruz, CA (US); Robert Shelansky, Santa Cruz, CA (US); Brandon Saint-John, Santa Cruz, CA (US)

(21) Appl. No.: 16/977,381

(22) PCT Filed: Feb. 28, 2019
(86) PCT No.: PCT/US19/20018

§ 371 Date: Sep. 1, 2020

Related U.S. Application Data
(60) Provisional application No. 62/637,277, filed on Mar. 1, 2018.

(51) Int. Cl. C12Q 1/6806; C12Q 1/6869

(52) U.S. Cl. CPC C12Q 1/6806; C12Q 1/6869; C12Q 2565/631; C12Q 2565/133; C12Q 2523/101

(57) ABSTRACT

Provided are methods of determining bound and unbound regions in nucleic acid molecules. In certain aspects, the methods include forming adducts in a double-stranded nucleic acid molecule that mark the locations of unbound regions in the double-stranded nucleic acid, and detecting the locations of the adducts in the double-stranded nucleic acid using a nanopore. Bound regions of the double-stranded nucleic acid molecule are determined based on the absence of adducts. In certain aspects, the double-stranded nucleic acid molecule is genomic DNA and the bound regions are nucleosome positions. As such, encompassed by the methods are methods of determining nucleosome positions in genomic DNA. Systems and kits that find use, e.g., in practicing the methods of the present disclosure are also provided.

### CROSS-REFERENCE TO RELATED APPLICATIONS

This application claims the benefit of U.S. Provisional Patent Application No. 62/637,277, filed Mar. 1, 2018, which application is incorporated herein by reference in its entirety.

### INTRODUCTION

In eukaryotic cells, DNA is packaged like a thread with distinct segments wrapped around molecular spools called nucleosomes. The location of these nucleosomes across a genome affects the accessibility of DNA and is essential for gene regulation. Current methods for investigating genome-wide positions of nucleosomes require fragmenting DNA into short 100-1000 bp segments. Buenrostro et al. (2015) Curr Protoc Mol Biol. 109: 21.29.1-9. As such, these methods do not permit profiling single-molecule nucleosome positions across an entire gene locus. The ability to phase nucleosome positions within one or more gene loci and flanking intergenic regions would enable the association of nucleosome positioning with gene expression and mRNA processing for a better understanding of gene regulation.

Recent advances in DNA sequencing have revolutionized the field of genomics, making it possible for even single research groups to generate large amounts of sequence data very rapidly and at a substantially lower cost. These high-throughput sequencing technologies make deep transcriptome sequencing and transcript quantification, whole genome sequencing and resequencing available to many more researchers and projects.

An emerging single molecule sequencing approach that has made significant progress in recent years is nanopore-based sequencing. Nanopore sequencing principally relies on the transition of DNA, RNA or individual nucleotides through a nanoscale-sized channel. A sequencing flow cell includes hundreds of independent micro-wells, each containing a bilayer perforated by nanopores. Sequencing is accomplished by measuring characteristic changes in current that are induced as the bases are threaded through the pore by a molecular motor protein. Library preparation is minimal, involving fragmentation of DNA and ligation of adapters, and can be done with or without PCR amplification. The library design may allow sequencing of both strands of DNA from a single molecule, which increases accuracy.

### SUMMARY

Provided are methods of determining bound and unbound regions in nucleic acid molecules. In certain aspects, the methods include forming adducts in a double-stranded nucleic acid molecule that mark the locations of unbound regions in the double-stranded nucleic acid, and detecting the locations of the adducts in the double-stranded nucleic acid using a nanopore. Bound regions of the double-stranded nucleic acid molecule are determined based on the absence of adducts. In certain aspects, the double-stranded nucleic acid molecule is genomic DNA and the bound regions are nucleosome positions. As such, encompassed by the methods are methods of determining nucleosome positions in genomic DNA. Systems and kits that find use, e.g., in practicing the methods of the present disclosure are also provided.

### BRIEF DESCRIPTION OF THE FIGURES

FIG. 1 illustrates a method of preparing a library for nanopore-based determination of nucleosome positions in genomic DNA according to one embodiment of the present disclosure. In this embodiment, the monoadduct-forming agent angelicin is employed.

FIG. 2 shows the structure of a monoadduct-forming agent (angelicin) which may be employed according to embodiments of the present disclosure.

FIG. 3 illustrates a method of preparing a library for nanopore-based determination of nucleosome positions in genomic DNA according to one embodiment of the present disclosure. In this embodiment, a psoralen diadduct-forming crosslinking agent is employed.

FIG. 4 shows an example diadduct-forming crosslinking reagent (panel A) and crosslinking approach (panel B) according to one embodiment of the present disclosure.

### DETAILED DESCRIPTION

Provided are methods of determining bound and unbound regions in nucleic acid molecules. In certain aspects, the methods include forming adducts in a double-stranded nucleic acid molecule that mark the locations of unbound regions in the double-stranded nucleic acid, and detecting the locations of the adducts in the double-stranded nucleic acid using a nanopore. Bound regions of the double-stranded nucleic acid molecule are determined based on the absence of adducts. In certain aspects, the double-stranded nucleic acid molecule is genomic DNA and the bound regions are nucleosome positions. As such, encompassed by the methods are methods of determining nucleosome positions in genomic DNA. Systems and kits that find use, e.g., in practicing the methods of the present disclosure are also provided.

Before the methods, systems and kits of the present disclosure are described in greater detail, it is to be understood that the methods, systems and kits are not limited to particular embodiments described, as such may, of course, vary. It is also to be understood that the terminology used herein is for the purpose of describing particular embodiments only, and is not intended to be limiting, since the scope of the methods, systems and kits will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods, systems and kits. The upper and lower limits of these smaller ranges may independently be included in the smaller ranges and are also encompassed within the methods, systems and kits, subject to any specifically excluded limit in the stated range. Where the stated range includes one or both of the limits, ranges excluding either or both of those included limits are also included in the methods, systems and kits.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods, systems and kits belong. Although any methods, systems and kits similar or equivalent to those described herein can also be used in the practice or testing of the methods, systems and kits, representative illustrative methods, systems and kits are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods, systems and kits are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods, systems and kits, which are, for clarity, described in the context of separate embodiments, may also be provided in combination in a single embodiment. Conversely, various features of the methods, systems and kits, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods, systems and kits and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Methods

As summarized above, the present disclosure provides methods of determining bound and unbound regions in nucleic acid molecules. In certain aspects, the double-stranded nucleic acid molecule is genomic DNA and the bound regions are nucleosome positions. As such, encompassed by the methods are methods of determining nucleosome positions in genomic DNA.

The methods and tools provided by the present disclosure overcome the barriers of current methods by taking advantage of recent nanopore sequencing technologies and certain adduct-forming agents, e.g., monoadduct-forming agents (e.g., angelicin) and diadduct-forming crosslinking reagents (e.g., psoralen-based crosslinking reagents) that from adducts in unbound DNA (e.g., genomic DNA not associated with nucleosomes—referred to herein as “linker genomic DNA”) and do not from adducts in bound DNA. Using nanopore sequencing technology, long DNA molecules (e.g., >10 kilobases (kb)) may be sequenced. With modified protocols, nanopore sequencing technology has been reported to produce N50 read lengths of ˜100 kb. Jain et al. (2017) bioRxiv. p. 128835. The methods further exploit the ability of nanopore sequencing to detect base modifications (see, e.g., Rand et al. (2017) Nature Methods 14:411-413; Simpson et al. (2017) Nature Methods 14:407-410), e.g., to identify monoadducts formed after alkali reversal of crosslinked DNA. The inventors have termed such methods “Add-Seq” (Adduct Sequencing).

In the subject methods for determining bound regions in a nucleic acid molecule, a “bound region” refers to a region of a double-stranded nucleic acid molecule which is bound by one or more nucleic acid-binding molecules. By virtue of being bound by the one or more nucleic acid-binding molecules, opposing strands of the nucleic acid molecule at the bound region are separated (e.g., not hybridized) or otherwise inaccessible to the adduct-forming agent employed, such that the adduct-forming agent does not form adducts at the bound region.

A “nucleic acid binding molecule” may be any molecule that binds to the nucleic acid molecule and prevents adduct formation of the nucleic acid molecule at the bound region. In some embodiments, a nucleic acid binding molecule is selected from the group consisting a polymer, a protein (e.g., a protein that includes a zinc finger domain, a protein that includes a helix-turn-helix domain, a protein that includes a leucine zipper domain, a transcription factor, a polymerase, a nuclease, a topoisomerase, a helicase, a chromatin protein (e.g., a histone protein) and/or the like), a nucleic acid, an aptamer, and a small molecule. In certain aspects, bound regions are produced from regions having nucleic acid binding molecules associated therewith, e.g., by treatment of such regions with a reagent. In one such example, the reagent is formaldehyde.

By “small molecule” is meant a compound having a molecular weight of 1000 atomic mass units (amu) or less. In some embodiments, the small molecule is 750 amu or less, 500 amu or less, 400 amu or less, 300 amu or less, or 200 amu or less. In certain aspects, the small molecule is not made of repeating molecular units such as are present in a polymer.

In the particular context of determining nucleosome positions in genomic DNA, the bound regions include, but are not limited to, the regions of the genomic DNA at which nucleosomes are positioned—that is, “nucleosome-associated genomic DNA”. The subject methods of determining nucleosome positions in genomic DNA exploit the protected/inaccessible nature of nucleosome-associated genomic DNA from the adduct-forming agent employed, such that adducts are not formed (or not substantially formed) in nucleosome-associated genomic DNA. A nucleosome is a basic unit of DNA packaging in eukaryotes, consisting of a segment of DNA wound in sequence around eight histone protein cores. The nucleosome core particle consists of approximately 146 base pairs (bp) of DNA wrapped in 1.67 left-handed superhelical turns around a histone octamer consisting of 2 copies each of the core histones H2A, H2B, H3, and H4. Genome-wide nucleosome positioning maps are available for many model organisms including mouse liver and brain (see Bargaje et al. (2012) Nucleic Acids Research 40(18):8965-78) and yeast (see Yuan et al. (2005) Science 309(5734):626-30).

The double-stranded nucleic acid molecule may be any double-stranded nucleic acid molecule of interest. By “double-stranded” is meant the nucleic acid molecule includes at least one hybridized region, e.g., by Watson-Crick base-pairing via complementarity between the opposing strands at the hybridized region. Hybridization may be intermolecular (e.g., double-stranded DNA, double-stranded RNA, double-stranded DNA-RNA hybrid molecules) or intramolecular, e.g., a single DNA or RNA strand that hybridizes to itself at one or more regions. In some embodiments, the double-stranded nucleic acid molecule is DNA. The DNA may be genomic DNA, mitochondrial DNA, cDNA, or the like. In some embodiments, the double-stranded nucleic acid molecule is RNA, such as an RNA molecule exhibiting intramolecular hybridization at one or more regions.

Nucleic acid molecules to be analyzed according to the methods of the present disclosure may be from a single cell, a plurality of cells (e.g., cultured cells), a tissue, an organ, or an organism (e.g., bacteria, yeast, or the like). In certain aspects, the nucleic acid molecules are from a cell(s), tissue, organ, and/or the like of an animal. In some embodiments, the animal is a mammal (e.g., a mammal from the genus Homo, a rodent (e.g., a mouse or rat), a dog, a cat, a horse, a cow, or any other mammal of interest). In certain aspects, the nucleic acid sample is from a cell(s), tissue, organ, and/or the like of a human. In other aspects, the nucleic acid sample is from a source other than a mammal, such as bacteria, yeast, insects (e.g., drosophila), amphibians (e.g., frogs (e.g., Xenopus)), viruses, plants, or any other non-mammalian source.

In some embodiments, the nucleic acid molecules are cell-free nucleic acid molecules, e.g., cell-free DNA, cell-free RNA, or both. Such cell-free nucleic acid molecules may present in, or obtained from, any suitable source. In certain aspects, the cell-free nucleic acid molecules are present in or obtained from a body fluid sample selected from whole blood, blood plasma, blood serum, amniotic fluid, saliva, urine, pleural effusion, bronchial lavage, bronchial aspirates, breast milk, colostrum, tears, seminal fluid, peritoneal fluid, pleural effusion, and stool. In some embodiments, the nucleic acid molecules are cell-free fetal DNAs. In certain aspects, the cell-free nucleic acid molecules are circulating tumor DNAs. In some embodiments, the cell-free nucleic acid molecules comprise infectious agent DNAs. In some embodiments, the cell-free nucleic acid molecules comprise DNAs from a transplant. The term “cell-free nucleic acid” as used herein can refer to nucleic acid isolated from a source having no cells or substantially no cells.

In certain aspects, the nucleic acid molecule is present in its native environment during exposure to the adduct-forming agent. For example, the nucleic acid molecule may be present in a cell (e.g., an intact cell or permeabilized cell) during exposure to the adduct-forming agent. In some embodiments, a cell-permeable adduct-forming agent that crosses an intact or permeabilized cell membrane may be employed. In certain aspects, the nucleic acid molecule is present in a cell lysate during exposure to the adduct-forming agent.

In other aspects, the nucleic acid molecule is part of a nucleic acid sample isolated from a cell(s), tissue, organ, and/or the like of an organism, e.g., an animal, such as a human. Approaches, reagents and kits for isolating, purifying and/or concentrating nucleic acid molecules from sources of interest are known in the art and commercially available. For example, kits for isolating DNA from a source of interest include the DNeasy®, RNeasy®, QlAamp®, QIAprep® and QIAquick® nucleic acid isolation/purification kits by Qiagen, Inc. (Germantown, Md.); the DNAzol®, ChargeSwitch®, Purelink®, GeneCatcher® nucleic acid isolation/purification kits by Life Technologies, Inc. (Carlsbad, Calif.); the NucleoMag®, NucleoSpin®, and NucleoBond® nucleic acid isolation/purification kits by Clontech Laboratories, Inc. (Mountain View, Calif.). In certain aspects, the nucleic acid is isolated from a fixed biological sample, e.g., formalin-fixed, paraffin-embedded (FFPE) tissue. Genomic DNA from FFPE tissue may be isolated using commercially available kits—such as the AllPrep® DNA/RNA FFPE kit by Qiagen, Inc. (Germantown, Md.), the RecoverAll® Total Nucleic Acid Isolation kit for FFPE by Life Technologies, Inc. (Carlsbad, Calif.), and the NucleoSpin® FFPE kits by Clontech Laboratories, Inc. (Mountain View, Calif.).

In some embodiments, forming adducts includes crosslinking the double-stranded nucleic acid molecule (e.g., genomic DNA) with a diadduct-forming crosslinking agent such that unbound regions are crosslinked and bound regions are not crosslinked, and reversing the crosslinks to form monoadducts from the diadducts. By “crosslinking” is meant covalently linking adjacent/opposite (e.g., complementary) strands of a double-stranded nucleic acid molecule, where the nucleic acid molecule may have two separate strands (e.g., genomic DNA, mitochondrial DNA, or the like) or a single strand, e.g., a single DNA or RNA strand having one or more regions of intramolecular hybridization.

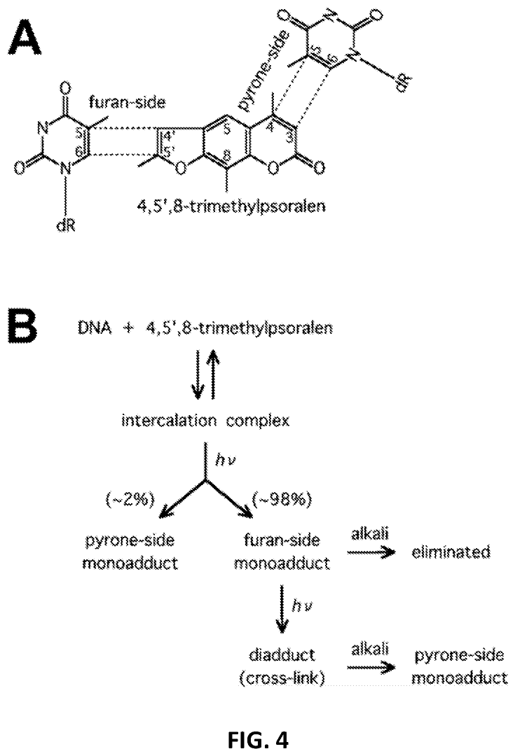

A crosslinking agent may be any crosslinking agent that crosslinks unbound regions of a double-stranded nucleic acid molecule of interest, where the crosslinks are such that, upon reversal of the crosslinks, a nanopore-detectable adduct (e.g., a monoadduct) remains that marks the locations of the former crosslinks in the unbound regions of the double-stranded nucleic acid molecule. A non-limiting example of a crosslinking agent meeting this criteria is a psoralen crosslinking agent. Psoralen crosslinking agents of interest include, but are not limited to, 4,5′,8-trimethylpsoralen (the structure of which is shown in FIG. 4, panel A, with possible bonds to thymine bases shown with dotted lines), 4′-aminomethyl-4,5′,8-trimethylpsoralen (AMT), 4′-(hydroxymethyl)-4,5′,8-trimethylpsoralen, 8-methoxypsoralen etc.

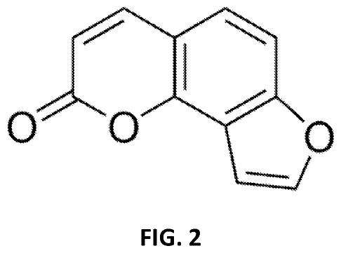

In some embodiments, forming monoadducts includes combining the double-stranded nucleic acid with a monoadduct-forming agent, and treating the double-stranded nucleic acid and the monoadduct-forming agent to form the monoadducts in the double-stranded nucleic acid. In some embodiments, treating the double-stranded nucleic acid and the monoadduct-forming agent includes exposing the double-stranded nucleic acid and the monoadduct-forming agent to ultraviolet light. According to some embodiments, the monoadduct-forming agent is a DNA intercalating agent. In certain embodiments, the DNA intercalating agent is a furanocoumarin compound. According to some embodiments, the furanocoumarin compound is an angular furanocoumarin compound. In certain embodiments, the angular furanocoumarin compound is angelicin. The structure of angelicin is shown in FIG. 2.

By “adduct” is meant a chemical moiety covalently attached to a nucleic acid molecule, which in some embodiments is a remnant, derivative, or the like, of a crosslink at an unbound region. A “diadduct” is a chemical moiety that crosslinks adjacent/opposite (e.g., complementary) strands at a location of a double-stranded nucleic acid molecule. A “monoadduct” is a chemical moiety covalently attached to only one strand at a location of a nucleic acid molecule. For example, in some embodiments of the methods of the present disclosure, a diadduct-forming crosslinking agent produces diadducts (or “crosslinks”) between adjacent/opposite (e.g., complementary) strands at unbound regions (e.g., linker genomic DNA) of a double-stranded nucleic acid molecule, and reversal of the crosslinks leaves monoadducts (e.g., pyrone-side monoadducts) at the unbound regions which are then detected using a nanopore.

Shown in FIG. 4, panel B, is an example procedure that may be employed to produce nanopore-detectable monoadducts in nucleic acid molecules of interest. In this particular example, 4,5′,8-trimethylpsoralen is employed as the crosslinking agent. As shown, DNA is contacted with 4,5′,8-trimethylpsoralen to produce reversible intercalation complexes. Irradiation of the intercalation complexes with UV light produces approximately 2% pyrone-side monoadducts and approximately 98% furan-side monoadducts. These monoadducts may be eliminated (e.g., by alkali treatment) or, upon continued or subsequent further exposure to UV light, converted to diadducts (crosslinks). In this example, the diadducts/crosslinks are reversed by alkali treatment (e.g., by contacting the cross-linked double-stranded nucleic acid molecule with an alkaline solution) to produce nanopore-detectable pyrone-side monoadducts. Due to the strong preference of 4,5′,8-trimethylpsoralen for addition on the furan-side (98%), adducts detected after alkali treatment will be enriched with those that were reversed from diadduct cross-links.

Forming adducts in a double-stranded nucleic acid molecule may be carried out using any of a variety of suitable approaches, which approaches may vary depending upon the adduct-forming agent employed. For example, forming adducts may be carried out by combining cells (e.g., intact or permeabilized cells) including double-stranded nucleic acid molecules of interest with a cell-permeable adduct-forming agent under conditions in which the adduct-forming agent crosses the cell membrane of the cells and forms adducts in the unbound regions of the double-stranded nucleic acid molecules of interest. As another example, forming adducts may be carried out by combining a cell lysate including double-stranded nucleic acid molecules of interest with a suitable adduct-forming agent under conditions in which the adduct-forming agent forms adducts in the unbound regions of the double-stranded nucleic acid molecules present in the lysate. In yet another example, forming adducts may be carried out by combining double-stranded nucleic acid molecules isolated from a source of interest (e.g., one or more cell(s), a tissue, organ, organism, environmental sample, or the like) with a suitable adduct-forming agent under conditions in which the adduct-forming agent forms adducts in the unbound regions of the double-stranded nucleic acid molecules present in the isolate. Regardless of the approach, the combining is carried out under conditions such that adducts are formed in the unbound regions, and adducts are not formed (or not substantially formed) in bound regions (e.g., nucleosome positions) of the double-stranded nucleic acid molecules. Such conditions may include, e.g., suitable selection of the concentration of adduct-forming agent, concentration of cells or double-stranded nucleic acid molecules of interest (e.g., present in a lysate or isolate), buffer, pH, temperature, and/or the like, which conditions may be selected based on the particular adduct-forming agent employed. When a photochemical adduct-forming agent is employed (e.g., angelicin, a psoralen crosslinking agent, or the like), the conditions further include irradiating the mixture of components with electromagnetic radiation having a wavelength suitable for the crosslinking agent. By way of example, a suitable wavelength for 4,5′,8-trimethylpsoralen is a wavelength in the ultraviolet range, including but not limited to a wavelength in the range of from 340 to 380 nm.

In certain aspects, methylation is used as an adduct. That is, in some embodiments, nucleosome positions are determined by detecting methyl groups using the nanopore. See Fatemi et al. (2005) Nucleic Acids Res. 33(20):e176.

Reversing crosslinks may be carried out using any of a variety of suitable approaches, which approaches may vary depending upon the nature of the crosslinks—as determined by, e.g., the diadduct-forming crosslinking agent employed. In certain aspects, reversing the crosslinks includes contacting the cross-linked double-stranded nucleic acid molecules with an alkaline solution.

In embodiments that include crosslinking the double-stranded nucleic acid, the methods may further include, subsequent to the crosslinking and prior to reversing the crosslinks, linking the ends of the double-stranded nucleic acid molecule. The ends may be linked, e.g., by ligating the ends directly to each other, ligating an adapter molecule to the ends such that the ends are linked via the adapter molecule, or the like. In certain aspects, linking the ends of the double-stranded nucleic acid molecule includes ligating a hairpin adapter molecule to the ends of the double-stranded nucleic acid molecule. Such an adapter molecule may include a component that facilitates subsequent cutting of the adapter molecule (e.g., subsequent to reversing the crosslinks) to unlink the ends of the double-stranded nucleic acid molecule, such as a recognition site for an endonuclease. As such, the methods may include, subsequent to reversing the crosslinks, cutting the hairpin adapter. In some embodiments, the hairpin adapter includes a uracil, and cutting the hairpin adapter includes excising the uracil from the hairpin adapter.

The double-stranded nucleic acid molecule may undergo any desired processing steps, such as processing steps useful for detecting the locations of the adducts in the double-stranded nucleic acid molecule using a nanopore. For example, the methods may include treating the ends of the double-stranded nucleic acid molecule to produce blunt ends. Blunting is a process by which a single-stranded overhang is either “filled in”, by the addition of nucleotides on the complementary strand using the overhang as a template for polymerization, or by “chewing back” the overhang, using an exonuclease activity. DNA polymerases, such as the Klenow fragment of DNA Polymerase I and T4 DNA Polymerase may be used to fill in (5′→3′) and chew back (3′→5′). Removal of a 5′ overhang can be accomplished with a nuclease, such as Mung Bean Nuclease.

Other processing steps useful for detecting the locations of the adducts in the double-stranded nucleic acid molecule using a nanopore may be employed. For example, the methods may include adding one or more nanopore sequencing adapters or subregions thereof to one or more ends of the double-stranded nucleic acid molecule. By “nanopore sequencing adapter” is meant one or more nucleic acid domains that include at least a portion of a nucleic acid sequence (or complement thereof) utilized by a nanopore sequencing platform of interest, such as a nanopore sequencing platform provided by Oxford Nanopore Technologies, e.g., a MinION™ GridIONx5™, PromethION™, or SmidgION™ nanopore-based sequencing system. Nanopore sequencing adapters of interest may be added via chemical or enzymatic ligation, or any other available approaches for joining one or more nucleic acid molecules to one or more ends of the double-stranded nucleic acid molecule. Suitable reagents (e.g., ligases) and kits for performing ligation reactions are known and available, e.g., the Instant Sticky-end Ligase Master Mix available from New England Biolabs (Ipswich, Mass.). Ligases that may be employed include, e.g., T4 DNA ligase (e.g., at low or high concentration), T4 DNA ligase, T7 DNA Ligase, E. coli DNA Ligase, Electro Ligase®, or the like. Conditions suitable for performing the ligation reaction will vary depending upon the type of ligase used.

In some embodiments, prior to detecting the locations of the adducts in the double-stranded nucleic acid molecule using a nanopore, the ends of the double-stranded nucleic acid molecule are covalently linked via a hairpin adapter that enables “2D” adduct detection (and, optionally, “2D” sequencing). By “2D” adduct detection in this context is meant adducts in both strands of the double-stranded nucleic acid molecule are detected as the strands (joined at their ends via the hairpin adapter) are translocated through the nanopore consecutively. A consensus of adduct locations may be obtained from the adduct locations detected during translocation of a first strand and adduct locations detected during translocation of the second strand (joined to the end of the first strand via the hairpin adapter), which consensus may be more accurate than the adduct locations detected from one strand alone. If sequencing is performed, “2D” sequencing in this context is meant both strands of the double-stranded nucleic acid molecule are sequenced as the strands (joined at their ends via the hairpin adapter) are translocated through the nanopore consecutively. A consensus sequence may be obtained from the sequence obtained from a first strand and the a sequence obtained from the second strand (joined to the end of the first strand via the hairpin adapter), which consensus sequence may be more accurate than a sequence obtained from one strand alone. In some embodiments, the methods include simultaneous “2D” adduct detection and “2D” sequencing.

In some embodiments, a nanopore sequencing adapter and a motor protein are attached to both ends of the double-stranded nucleic acid molecule. The sequencing adapter has a molecule attached to it (e.g., cholesterol or other suitable molecule) that promotes binding of the double-stranded nucleic acid molecule to the nanopore membrane. After one side is translocated through the pore, the molecule (e.g., cholesterol) helps keep the DNA molecule near the opening of the pore, and stochastically the other strand of the double-stranded nucleic acid molecule is pulled through. Adduct and/or sequencing data from the two strands may then be used to produce consensus adduct locations and/or a consensus sequence.

As summarized above, the methods for determining bound regions in a double-stranded nucleic acid molecule include detecting the locations of the adducts in the double-stranded nucleic acid molecule using a nanopore. By “detecting the locations of the adducts” is meant detecting at least a portion of the adducts in at least a portion of one or both strands of the double-stranded nucleic acid molecule. As will be appreciated, at this stage, the strands of the “double-stranded nucleic acid molecule” may have been separated from one another and the detection may involve detecting the locations of adducts in only one of the two strands, detecting the locations of adducts in both strands in parallel (e.g., using two individually-addressable nanopores—one individually-addressable nanopore for each strand), detecting the locations of adducts in both strands sequentially (e.g., by “2D” adduct detection, etc.), and/or the like.

In certain aspects, detecting the locations of the adducts in the double-stranded nucleic acid molecule using a nanopore includes applying a potential difference across the nanopore, exposing one or both strands of the double-stranded nucleic acid molecule to the nanopore in a sequential manner, and detecting electrical signals from the nanopore corresponding to the adducts in the double-stranded nucleic acid molecule. In some embodiments, the rate at which one or both strands are exposed to the nanopore is controlled using a processive enzyme. Non-limiting examples of processive enzymes that may be employed include polymerases (e.g., a phi29 or other suitable polymerase) and helicases, e.g., a He1308 helicase, a RecD helicase, a Tral helicase, a Tral subgroup helicase, an XPD helicase, or the like. One or both strands of the double-stranded nucleic acid molecule may be bound by the processive enzyme (e.g., by binding of the processive enzyme to a recognition site present in a sequencing adapter ligated to one or both strands), followed by the resulting complex being drawn to the nanopore, e.g., by a potential difference applied across the nanopore. In other aspects, the processive enzyme may be located at the nanopore (e.g., attached to or adjacent to the nanopore) such that the processive enzyme binds one or both strands of the double-stranded nucleic acid molecule upon arrival of the one or both strands at the nanopore.

In some embodiments, exposing one or both strands of the double-stranded nucleic acid molecule to the nanopore in a sequential manner includes translocating at least a portion of one or both strands of the double-stranded nucleic acid molecule through the nanopore. For example, the detecting may include delivering one or both strands of the double-stranded nucleic acid molecule to a nanopore (or an enzyme (e.g., a processive enzyme) located at or near the nanopore), translocating a strand through the nanopore (unzipping a second hybridized strand, if present) and detecting electrical signals from the nanopore corresponding to the adducts during the translocation.

In certain aspects, the locations of adducts are detected in a contiguous stretch of a strand of the double-stranded nucleic acid molecule of 500 bases or greater, 1 kilobase (kb) or greater, 2 kb or greater, 3 kb or greater, 4 kb or greater, 5 kb or greater, 6 kb or greater, 7 kb or greater, 8 kb or greater, 9 kb or greater, 10 kb or greater, 15 kb or greater, 20 kb or greater, 25 kb or greater, 30 kb or greater, 35 kb or greater, 40 kb or greater, 45 kb or greater, 50 kb or greater, 55 kb or greater, 60 kb or greater, 65 kb or greater, 70 kb or greater, 75 kb or greater, 80 kb or greater, 85 kb or greater, 90 kb or greater, 95 kb or greater, or 100 kb or greater.

Computational approaches (e.g., in the form of software) may be employed to detect the locations of the adducts in the double-stranded nucleic acid molecule using the nanopore, determine bound regions in the double-stranded nucleic acid molecule based on the detected locations of adducts, sequence the double-stranded nucleic acid molecule using the nanopore, etc., and any combinations thereof.

In some embodiments, a computational approach includes a modified Hidden Markov Model and/or a modified Dirchlet process mixture model as employed in the context of nanopore-based methylation detection. See, e.g., Rand et al. (2017) Nature Methods 14:411-413; Simpson et al. (2017) Nature Methods 14:407-410.

In certain aspects, a machine learning (or “deep learning”) computational approach is employed. Such approaches may include one or more aspects of such learning described in Teng et al. (2017) “Translating nanopore raw signal directly into nucleotide sequence using deep learning” bioRxiv doi: https://doi.org/10.1101/179531; and Stoiber et al. (2017) “De novo Identification of DNA Modifications Enabled by Genome-Guided Nanopore Signal Processing” bioRxiv doi:http://dx.doi.org/10.1101/094672.

In certain aspects, synthetic sequences with known locations modified by an adduct (e.g., a psoralen adduct) are used in order to determine the adduct (e.g., psoralen) positions on the nucleic acid molecule in the absence of a control that has not been treated with the crosslinking agent.

In some embodiments, in addition to (e.g., in parallel with) detecting electrical signals from the nanopore corresponding to the adducts, the methods include sequencing at least a portion of one or both strands of the double-stranded nucleic acid molecule using the nanopore. Details regarding nanopore-based sequencing are described, e.g., in Feng et al. (2015) Genomics, Proteomics & Bioinformatics 13(1):4-16. Any of the nanopore-based sequencing embodiments described herein may be carried out using, e.g., a MinION™, GridIONx5™, PromethION™, or SmidgION™ nanopore-based sequencing system, available from Oxford Nanopore Technologies. Detailed design considerations and protocols for carrying out the sequencing are provided with such systems.

As summarized above, encompassed by the methods for determining bound regions in double-stranded nucleic acid molecules are methods for determining nucleosome positions in genomic DNA. Such methods exploit the protected/inaccessible nature of nucleosome-associated genomic DNA from the adduct-forming agent employed, such that adducts are not formed (or not substantially formed) in nucleosome-associated genomic DNA. The methods include forming adducts in genomic DNA that mark the locations of linker genomic DNA in the genomic DNA, and detecting the locations of the adducts in the genomic DNA using a nanopore. The nucleosome positions in the genomic DNA are determined based on the absence of adducts.

The methods for determining nucleosome positions in genomic DNA may implement any of the steps, reagents, tools, etc. described above in the more general context of determining bound regions in double-stranded nucleic acid molecules. Determining nucleosome positions in genomic DNA, based on the detected locations of the adducts, where nucleosome positions in the genomic DNA are determined based on the absence of adducts, may be carried out using a computational method (e.g., in the form of software) and includes any of the computational approaches described herein.

The methods for determining nucleosome positions may further include assessing mRNA processing in the source of the genomic DNA (e.g., one or more cells, a tissue, an organ, etc.) based on the determined nucleosome positions in the genomic DNA. Assessing mRNA processing may include assessing the transcriptional regulatory status (e.g., gene expression) of one or more genes present in the genomic DNA based on the determined nucleosome positions, assessing mRNA splicing based on the determined nucleosome positions, and the like, including any combinations thereof. It has been shown that nucleosome positioning affects mRNA processing. See, e.g., Andersson et al. (2009) Genome Res. 19:1732-1741.

By way of example, the inventors have determined that lung cancer-associated mutations in the gene encoding SMARC4 (a subunit of the SWI/SNF remodeling complex that can shift positions of nucleosomes in an ATP-dependent manner) correspond to significant changes in splicing through exon skipping. The methods for determining nucleosome positions may include determining alterations in nucleosome positions in a genomic region in a diseased state (e.g., cancer) versus a normal/healthy state. Current methods for genome-wide nucleosome profiling are inadequate for making such determinations due to the long distances between promoters and affected exons. In contrast, the present methods enable detection and quantification of nucleosome positions across long stretches (>10 kb) of single DNA molecules, in turn enabling determination of nucleosome positions on a single molecule of genomic DNA at promoters and within the gene body. Accordingly, encompassed by the subject methods for determining nucleosome positions are methods for assessing differential nucleosome occupancy or phasing near the promoters of genes, differential occupancy or phasing on affected exons, and combinations thereof.

An example approach for determining nucleosome positions in accordance with the methods of the present disclosure is schematically illustrated in FIG. 2. In this particular example, cells of interest are treated with angelicin and, after irradiation with UV light, linker DNA (DNA not associated with nucleosomes) will be marked with monoadducts that may be detected using a nanopore. The nucleosome-associated DNA will not be marked with such monoadducts, however, because it is protected from the angelicin.

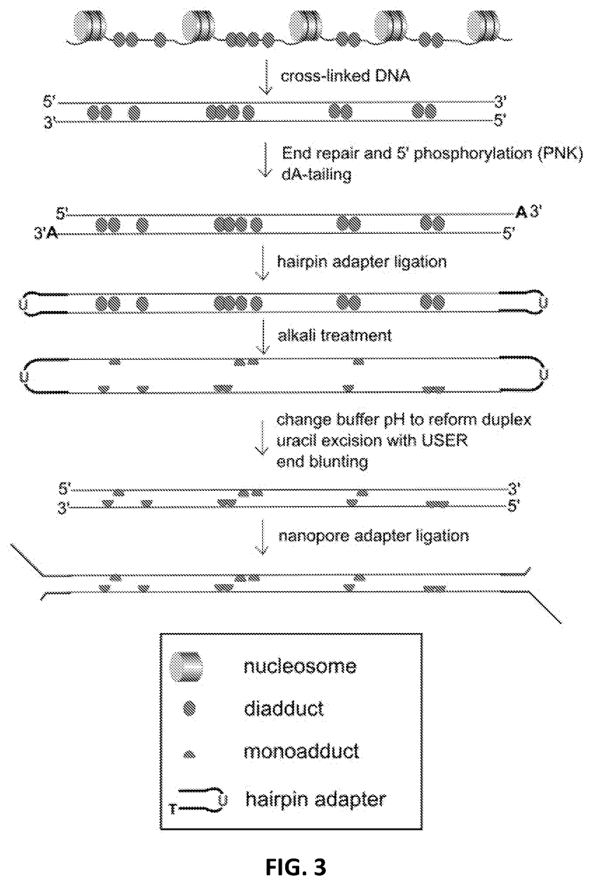

A further example approach for determining nucleosome positions in accordance with the methods of the present disclosure is schematically illustrated in FIG. 3. In this particular example, cells of interest are treated with 4,5′,8-trimethylpsoralen and, after irradiation with UV light, linker DNA (DNA not associated with nucleosomes) will crosslink to form diadducts with pyrimidines on adjacent strands of DNA. The nucleosome-associated DNA will not be crosslinked, however, because it is protected from the crosslinking agent.

### Systems

As summarized above, the present disclosure also provides systems. The systems find use in a variety of applications, including, e.g., practicing any of the methods of the present disclosure, including carrying out one or more of any of the steps described above in the Methods section of the present disclosure.

In certain aspects, provided is a system that includes a device including a substrate having a nanopore therein, the substrate separating a first fluid chamber from a second fluid chamber. The device further includes a power source electrically coupled to electrodes, where the power source and electrodes are adapted to apply a potential difference between the first fluid chamber and the second fluid chamber. The system further includes instructions that cause the system to apply a potential difference between the first fluid chamber and the second fluid chamber such that one or both strands of a double-stranded nucleic acid molecule in the first fluid chamber is drawn toward the second fluid chamber and exposed to the nanopore in a sequential manner, where the double-stranded nucleic acid molecule includes adducts that mark the locations of unbound regions of the double-stranded nucleic acid molecule. The instructions further cause the system to detect electrical signals from the nanopore corresponding to the adducts, and record the locations of the adducts in the double-stranded nucleic acid molecule. In certain aspects, the instructions further cause the system to sequence at least a portion of the double-stranded nucleic acid molecule using the nanopore.

Encompassed by the systems that find use in determining bound regions in a double-stranded nucleic acid molecule are systems that find use in determining nucleosome positions in genomic DNA. The instructions for such systems cause the system to apply a potential difference between the first fluid chamber and the second fluid chamber such that genomic DNA in the first fluid chamber is drawn toward the second fluid chamber and exposed to the nanopore in a sequential manner, where the genomic DNA includes adducts that mark the locations of linker genomic DNA in the genomic DNA. The instructions for such systems further cause the system to detect electrical signals from the nanopore corresponding to the adducts, and record the locations of the adducts in the genomic DNA. The instructions for such systems may further cause the system to assess mRNA processing in the source of the genomic DNA (e.g., one or more cells, a tissue, an organ, etc.) based on the determined nucleosome positions in the genomic DNA. Assessing mRNA processing may include assessing the transcriptional regulatory status (e.g., gene expression) of one or more genes present in the genomic DNA based on the determined nucleosome positions, assessing mRNA splicing based on the determined nucleosome positions, and the like, including any combinations thereof. The instructions for such systems may further cause the system to assess differential nucleosome occupancy or phasing near the promoters of genes, differential occupancy or phasing on affected exons, and combinations thereof.

The systems may be adapted (e.g., include instructions) to detect electrical signals from the nanopore corresponding to the adducts in a contiguous stretch of the genomic DNA of 500 bases or greater, 1 kilobase (kb) or greater, 2 kb or greater, 3 kb or greater, 4 kb or greater, 5 kb or greater, 6 kb or greater, 7 kb or greater, 8 kb or greater, 9 kb or greater, 10 kb or greater, 15 kb or greater, 20 kb or greater, 25 kb or greater, 30 kb or greater, 35 kb or greater, 40 kb or greater, 45 kb or greater, 50 kb or greater, 55 kb or greater, 60 kb or greater, 65 kb or greater, 70 kb or greater, 75 kb or greater, 80 kb or greater, 85 kb or greater, 90 kb or greater, 95 kb or greater, or 100 kb or greater, and record the locations of such adducts.

The systems of the present disclosure may include a processive enzyme for controlling the rate at which one or both strands are exposed to the nanopore. Non-limiting examples of processive enzymes that may be employed include polymerases (e.g., a phi29 or other suitable polymerase) and helicases, e.g., a He1308 helicase, a RecD helicase, a Tral helicase, a Tral subgroup helicase, an XPD helicase, or the like.

In some embodiments, the device is a commercially available nanopore sequencing device, such as a MinION™, GridIONx5™, PromethION™, or SmidgION™ nanopore-based sequencing device available from Oxford Nanopore Technologies.

The present disclosure includes computer-readable medium, including non-transitory computer-readable medium, which stores instructions for methods, or portions thereof, described herein, and which may be part of the systems of the present disclosure. Aspects of the present disclosure include computer-readable medium storing instructions that, when executed, cause the system to perform one or more steps of a method as described herein.

In some embodiments, instructions in accordance with the methods and systems described herein can be coded onto a computer-readable medium in the form of “programming”, where the term “computer-readable medium” as used herein refers to any storage or transmission medium that participates in providing instructions and/or data to a computer for execution and/or processing. Examples of storage media include a floppy disk, hard disk, optical disk, magneto-optical disk, CD-ROM, CD-R, magnetic tape, non-volatile memory card, ROM, DVD-ROM, Blue-ray disk, solid state disk, and network attached storage (NAS), whether or not such devices are internal or external to the computer. A file containing information can be “stored” on computer-readable medium, where “storing” means recording information such that it is accessible and retrievable at a later date by a computer.

Any steps of the methods or those carried out by the systems of the present disclosure can be executed using programming that can be written in one or more of any number of computer programming languages. Such languages include, for example, Java (Sun Microsystems, Inc., Santa Clara, Calif.), Visual Basic (Microsoft Corp., Redmond, Wash.), and C++ (AT&T Corp., Bedminster, N.J.), as well as any many others.

### Kits

As summarized above, the present disclosure provides kits. The kits may include any reagents, devices, instructions (e.g., present on one or more non-transitory computer-readable medium), etc. useful for practicing the methods of the present disclosure, including any reagents, devices, instructions, etc. described above in the Methods and Systems sections of the present disclosure.

In some embodiments, provided is a kit that includes an adduct-forming agent that forms adducts in a double-stranded nucleic acid molecule that mark the locations of unbound regions of the double-stranded nucleic acid molecule, and instructions for using the adduct-forming agent in a method for determining bound regions in a double-stranded nucleic acid molecule by detecting the locations of adducts in the double-stranded nucleic acid molecule using a nanopore.

In some embodiments, the adduct-forming agent is a monoadduct-forming agent, e.g., a DNA intercalating agent. In certain embodiments, the DNA intercalating agent is a furanocoumarin compound. According to some embodiments, the furanocoumarin compound is an angular furanocoumarin compound. In certain embodiments, the angular furanocoumarin compound is angelicin. In certain aspects, the adduct-forming agent is a diadduct-forming crosslinking agent, such as a psoralen crosslinking agent, a non-limiting example of which is 4,5′,8-trimethylpsoralen. The kits may include additional reagents, such as a crosslink-reversing reagent, a non-limiting example of which is an alkali solution. In some embodiments, the kits include one or more adapter molecules for linking the ends of double-stranded nucleic acid molecules. The adapter molecule may be a hairpin adapter molecule, and the instructions may include instructions for linking the ends of a crosslinked double-stranded nucleic acid molecule using the hairpin adapter molecule. The kits of the present disclosure may include one or more nanopore sequencing adapters that facilitate analysis of one or both strands of the double-stranded DNA molecules on a nanopore sequencing device. The kits may include reagents such as ligases, ligase buffers, etc. useful for linking adapter molecules to the double-stranded nucleic acid molecules.

Components of the kits may be present in separate containers, or multiple components may be present in a single container. A suitable container includes a single tube (e.g., vial), one or more wells of a plate (e.g., a 96-well plate, a 384-well plate, etc.), or the like.

The kits may include instructions, e.g., for using the adduct-forming agent in a method for determining bound regions in a double-stranded nucleic acid molecule by detecting the locations of adducts in the double-stranded nucleic acid molecule using a nanopore. In some embodiments, the kits include instructions for using the adduct-forming agent in a method for determining nucleosome positions in genomic DNA by detecting the locations of adducts in the genomic DNA using a nanopore.

The instructions may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

Notwithstanding the appended claims, the present disclosure is also defined by the following clauses:

1. A method for determining nucleosome positions in genomic DNA, comprising:
forming adducts in genomic DNA that mark the locations of linker genomic DNA in the genomic DNA; and
detecting the locations of the adducts in the genomic DNA using a nanopore,
wherein nucleosome positions in the genomic DNA are determined based on the absence of adducts.
   2. The method according to clause 1, wherein forming adducts in the genomic DNA comprises forming monoadducts in the genomic DNA.
   3. The method according to clause 2, wherein forming monoadducts in the genomic DNA comprises combining the genomic DNA with a monoadduct-forming agent, and treating the genomic DNA and the monoadduct-forming agent to form the monoadducts in the genomic DNA.
   4. The method according to clause 3, wherein treating the genomic DNA and the monoadduct-forming agent comprises exposing the genomic DNA and the monoadduct-forming agent to ultraviolet light.
   5. The method according to clause 4, wherein the monoadduct-forming agent is a DNA intercalating agent.
   6. The method according to clause 5, wherein the DNA intercalating agent is a furanocoumarin compound.
   7. The method according to clause 6, wherein the furanocoumarin compound is an angular furanocoumarin compound.
   8. The method according to clause 7, wherein the angular furanocoumarin compound is angelicin.
   9. The method according to clause 2, wherein forming monoadducts in the genomic DNA comprises:
crosslinking the genomic DNA with a diadduct-forming crosslinking agent such that linker genomic DNA is crosslinked and nucleosome-associated genomic DNA is not crosslinked; and
reversing the crosslinks to form monoadducts from the diadducts.
   10. The method according to clause 9, wherein the diadduct-forming crosslinking agent is a furanocoumarin crosslinking agent.
   11. The method according to clause 10, wherein the furanocoumarin crosslinking agent is a linear furanocoumarin crosslinking agent.
   12. The method according to clause 11, wherein the linear furanocoumarin crosslinking agent is a psoralen crosslinking agent.
   13. The method according to clause 12, wherein the psoralen crosslinking agent is 4,5′,8-trimethylpsoralen.
   14. The method according to any one of clauses 9 to 13, wherein the genomic DNA is present in a cell during the crosslinking.
   15. The method according to any one of clauses 9 to 14, further comprising, subsequent to the crosslinking and prior to reversing the crosslinks, linking the ends of the genomic DNA.
   16. The method according to clause 15, wherein linking the ends of the genomic DNA comprises ligating a hairpin adapter molecule to the ends of the genomic DNA.
   17. The method according to clause 16, wherein subsequent to reversing the crosslinks, cutting the hairpin adapter.
   18. The method according to clause 17, wherein the hairpin adapter comprises a uracil, and wherein cutting the hairpin adapter comprises excising the uracil from the hairpin adapter.
   19. The method according to any one of clauses 9 to 18, wherein reversing the crosslinks comprises contacting the cross-linked linker genomic DNA with an alkaline solution.
   20. The method according to any one of clauses 9 to 19, wherein subsequent to reversing the crosslinks, treating the ends of the genomic DNA to produce blunt ends.
   21. The method according to any one of clauses 1 to 20, wherein prior to detecting the locations of the adducts in the genomic DNA using a nanopore, adding one or more nanopore sequencing adapters to one or more ends of the genomic DNA.
   22. The method according to any one of clauses 1 to 21, wherein detecting the locations of the adducts in the genomic DNA using a nanopore comprises:
applying a potential difference across the nanopore;
exposing one or both strands of the genomic DNA to the nanopore in a sequential manner; and
detecting electrical signals from the nanopore corresponding to the adducts in the genomic DNA.
   23. The method according to clause 22, wherein a processive enzyme controls the rate of exposure of one or both strands of the genomic DNA to the nanopore in the sequential manner.
   24. The method according to clause 22 or clause 23, wherein exposing one or both strands of the genomic DNA to the nanopore in a sequential manner comprises translocating at least a portion of one or both strands of the genomic DNA through the nanopore.
   25. The method according to any one of clauses 1 to 24, wherein the locations of adducts are detected in a contiguous stretch of genomic DNA of 5 kilobases (kb) or greater.
   26. The method according to clause 25, wherein the locations of adducts are detected in a contiguous stretch of genomic DNA of 10 kb or greater.
   27. The method according to any one of clauses 1 to 26, further comprising sequencing at least a portion of the genomic DNA using the nanopore.
   28. The method according to any one of clauses 1 to 27, wherein nucleosome positions in the genomic DNA are determined using a computational method.
   29. The method according to any one of clauses 1 to 28, further comprising assessing mRNA processing in the source of the genomic DNA based on the nucleosome positions in the genomic DNA.
   30. The method according to any one of clauses 1 to 29, wherein the genomic DNA is yeast genomic DNA.
   31. The method according to any one of clauses 1 to 29, wherein the genomic DNA is mammalian genomic DNA.
   32. The method according to clause 31, wherein the genomic DNA is tumor genomic DNA.
   33. A system, comprising:
a device, comprising:
   a substrate having a nanopore therein, the substrate separating a first fluid chamber from a second fluid chamber;
   a power source electrically coupled to electrodes, wherein the power source and electrodes are adapted to apply a potential difference between the first fluid chamber and the second fluid chamber; and
instructions that cause the system to:
   apply a potential difference between the first fluid chamber and the second fluid chamber such that genomic DNA in the first fluid chamber is drawn toward the second fluid chamber and exposed to the nanopore in a sequential manner, wherein the genomic DNA comprises adducts that mark the locations of linker genomic DNA in the genomic DNA;
   detect electrical signals from the nanopore corresponding to the adducts; and
   record the locations of the adducts in the genomic DNA.
      34. The system of clause 33, wherein the instructions further cause the system to sequence at least a portion of the genomic DNA using the nanopore.
      35. The system of clause 33 or clause 34, wherein the instructions cause the system to detect electrical signals from the nanopore corresponding to the adducts in a contiguous stretch of the genomic DNA of 5 kilobases (kb) or greater.
      36. The system of clause 35, wherein the instructions cause the system to detect electrical signals from the nanopore corresponding to the adducts in a contiguous stretch of the genomic DNA of 10 kb or greater.
      37. The system of any one of clauses 33 to 36, wherein exposing the genomic DNA to the nanopore in a sequential manner comprises translocating at least a portion of the genomic DNA through the nanopore.
      38. The system of any one of clauses 33 to 37, comprising a processive enzyme that controls the rate of exposure of the genomic DNA to the nanopore in the sequential manner.
      39. The system of any one of clauses 33 to 38, wherein the instructions cause the system to determine nucleosome positions in the genomic DNA based on the absence of adducts.
      40. The system of clause 39, wherein the instructions cause the system to assess mRNA processing in the source of the genomic DNA based on the nucleosome positions in the genomic DNA.

41. A kit, comprising:
an adduct-forming agent that forms adducts in genomic DNA that mark the locations of linker genomic DNA in the genomic DNA; and
instructions for using the adduct-forming agent in a method for determining nucleosome positions in genomic DNA by detecting the locations of adducts in the genomic DNA using a nanopore.
   42. The kit of clause 41, wherein the adduct-forming agent is a monoadduct-forming agent.
   43. The kit of clause 42, wherein the monoadduct-forming agent is a DNA intercalating agent.
   44. The kit of clause 43, wherein the DNA intercalating agent is a furanocoumarin compound.
   45. The kit of clause 44, wherein the furanocoumarin compound is an angular furanocoumarin compound.
   46. The kit of clause 45, wherein the angular furanocoumarin compound is angelicin.
   47. The kit of clause 41, wherein the adduct-forming agent is a diadduct-forming crosslinking agent.
   48. The kit of clause 47, wherein the diadduct-forming crosslinking agent is a furanocoumarin crosslinking agent.
   49. The kit of clause 48, wherein the furanocoumarin crosslinking agent is a linear furanocoumarin crosslinking agent.
   50. The kit of clause 49, wherein the linear furanocoumarin crosslinking agent is a psoralen crosslinking agent.
   51. The kit of clause 50, wherein the psoralen crosslinking agent is 4,5′,8-trimethylpsoralen.
   52. The kit of any one of clauses 47 to 51, further comprising a crosslink-reversing reagent.
   53. The kit of clause 52, wherein the crosslink-reversing reagent is an alkali crosslink-reversing reagent.
   54. The kit of any one of clauses 47 to 53, further comprising a hairpin adapter molecule, and wherein the instructions comprise instructions for linking the ends of crosslinked genomic DNA using the hairpin adapter molecule.
   55. The kit of any one of clauses 41 to 54, further comprising a nanopore sequencing adapter molecule to facilitate detection of the locations of the adducts in the genomic DNA using a nanopore.
   56. A method for determining bound regions in a double-stranded nucleic acid molecule, comprising:
forming adducts in a double-stranded nucleic acid molecule that mark the locations of unbound regions in the double-stranded nucleic acid; and
detecting the locations of the adducts in the double-stranded nucleic acid using a nanopore,
wherein bound regions of the double-stranded nucleic acid molecule are determined based on the absence of adducts.
   57. The method according to clause 56, wherein forming adducts in the double-stranded nucleic acid comprises forming monoadducts in the double-stranded nucleic acid.
   58. The method according to clause 57, wherein forming monoadducts in the double-stranded nucleic acid comprises combining the double-stranded nucleic acid with a monoadduct-forming agent, and treating the double-stranded nucleic acid and the monoadduct-forming agent to form the monoadducts in the double-stranded nucleic acid.
   59. The method according to clause 58, wherein treating the double-stranded nucleic acid and the monoadduct-forming agent comprises exposing the double-stranded nucleic acid and the monoadduct-forming agent to ultraviolet light.
   60. The method according to clause 58 or 59, wherein the monoadduct-forming agent is a DNA intercalating agent.
   61. The method according to clause 60, wherein the DNA intercalating agent is a furanocoumarin compound.
   62. The method according to clause 61, wherein the furanocoumarin compound is an angular furanocoumarin compound.
   63. The method according to clause 62, wherein the angular furanocoumarin compound is angelicin.
   64. The method according to clause 57, wherein forming monoadducts in the double-stranded nucleic acid comprises:
crosslinking the double-stranded nucleic acid with a diadduct-forming crosslinking agent such that unbound double-stranded nucleic acid is crosslinked and bound double-stranded nucleic acid is not crosslinked; and
reversing the crosslinks to form monoadducts from the diadducts.
   65. The method according to clause 64, wherein the diadduct-forming crosslinking agent is a furanocoumarin crosslinking agent.
   66. The method according to clause 65, wherein the furanocoumarin crosslinking agent is a linear furanocoumarin crosslinking agent.
   67. The method according to clause 66, wherein the linear furanocoumarin crosslinking agent is a psoralen crosslinking agent.
   68. The method according to clause 67, wherein the psoralen crosslinking agent is 4,5′,8-trimethylpsoralen.
   69. The method according to any one of clauses 64 to 68, wherein the double-stranded nucleic acid molecule is present in a cell during the crosslinking.
   70. The method according to any one of clauses 64 to 69, further comprising, subsequent to the crosslinking and prior to reversing the crosslinks, linking the ends of the double-stranded nucleic acid molecule.
   71. The method according to clause 70, wherein linking the ends of the double-stranded nucleic acid molecule comprises ligating a hairpin adapter molecule to the ends of the double-stranded nucleic acid molecule.
   72. The method according to clause 71, wherein subsequent to reversing the crosslinks, cutting the hairpin adapter.
   73. The method according to clause 72, wherein the hairpin adapter comprises a uracil, and wherein cutting the hairpin adapter comprises excising the uracil from the hairpin adapter.
   74. The method according to any one of clauses 64 to 73, wherein reversing the crosslinks comprises contacting the cross-linked double-stranded nucleic acid molecule with an alkaline solution.
   75. The method according to any one of clauses 64 to 74, wherein subsequent to reversing the crosslinks, treating the ends of the double-stranded nucleic acid molecule to produce blunt ends.
   76. The method according to any one of clauses 56 to 74, wherein prior to detecting the locations of the adducts in the double-stranded nucleic acid molecule using a nanopore, adding one or more nanopore sequencing adapters to one or more ends of the double-stranded nucleic acid molecule.
   77. The method according to any one of clauses 56 to 76, wherein detecting the locations of the adducts in the double-stranded nucleic acid molecule using a nanopore comprises:
applying a potential difference across the nanopore;
exposing one or both strands of the double-stranded nucleic acid molecule to the nanopore in a sequential manner; and
detecting electrical signals from the nanopore corresponding to the adducts in the double-stranded nucleic acid molecule.
   78. The method according to clause 77, wherein a processive enzyme controls the rate of exposure of one or both strands of the double-stranded nucleic acid molecule to the nanopore in the sequential manner.
   79. The method according to clause 77 or clause 78, wherein exposing one or both strands of the double-stranded nucleic acid molecule to the nanopore in a sequential manner comprises translocating at least a portion of one or both strands of the double-stranded nucleic acid molecule through the nanopore.
   80. The method according to any one of clauses 56 to 79, wherein the locations of adducts are detected in a contiguous stretch of a strand of the double-stranded nucleic acid molecule of 1 kilobase (kb) or greater.
   81. The method according to clause 80, wherein the locations of adducts are detected in a contiguous stretch of a strand of the double-stranded nucleic acid molecule of 3 kb or greater.
   82. The method according to any one of clauses 56 to 81, further comprising sequencing at least a portion of one or both strands of the double-stranded nucleic acid molecule using the nanopore.
   83. The method according to any one of clauses 56 to 82, wherein bound regions in the double-stranded nucleic acid molecule are determined using a computational method.
   84. The method according to any one of clauses 56 to 83, wherein the double-stranded nucleic acid molecule is double-stranded DNA.
   85. The method according to any one of clauses 56 to 83, wherein the double-stranded nucleic acid molecule is an RNA strand having secondary structure.
   86. A system, comprising:
a device, comprising:
   a substrate having a nanopore therein, the substrate separating a first fluid chamber from a second fluid chamber;
   a power source electrically coupled to electrodes, wherein the power source and electrodes are adapted to apply a potential difference between the first fluid chamber and the second fluid chamber; and
instructions that cause the system to:
   apply a potential difference between the first fluid chamber and the second fluid chamber such that a double-stranded nucleic acid molecule in the first fluid chamber is drawn toward the second fluid chamber and exposed to the nanopore in a sequential manner, wherein the double-stranded nucleic acid molecule comprises adducts that mark the locations of unbound regions of the double-stranded nucleic acid molecule;
   detect electrical signals from the nanopore corresponding to the adducts; and
   record the locations of the adducts in the double-stranded nucleic acid molecule.
      87. The system of clause 86, wherein the instructions further cause the system to sequence at least a portion of the double-stranded nucleic acid molecule using the nanopore.
      88. The system of clause 86 or clause 87, wherein exposing the double-stranded nucleic acid molecule to the nanopore in a sequential manner comprises translocating at least a portion of the double-stranded nucleic acid molecule through the nanopore.
      89. The system of any one of clauses 86 to 88, comprising a processive enzyme that controls the rate of exposure of the double-stranded nucleic acid molecule to the nanopore in the sequential manner.
      90. The system of any one of clauses 86 to 89, wherein the instructions cause the system to determine bound regions in the double-stranded nucleic acid molecule based on the absence of adducts.
      91. A kit, comprising:
an adduct-forming agent that forms adducts in a double-stranded nucleic acid molecule that mark the locations of unbound regions of the double-stranded nucleic acid molecule; and
instructions for using the adduct-forming agent in a method for determining bound regions in a double-stranded nucleic acid molecule by detecting the locations of adducts in the double-stranded nucleic acid molecule using a nanopore.
   92. The kit of clause 91, wherein the adduct-forming agent is a monoadduct-forming agent.
   93. The kit of clause 92, wherein the monoadduct-forming agent is a DNA intercalating agent.
   94. The kit of clause 93, wherein the DNA intercalating agent is a furanocoumarin compound.
   95. The kit of clause 94, wherein the furanocoumarin compound is an angular furanocoumarin compound.
   96. The kit of clause 95, wherein the angular furanocoumarin compound is angelicin.
   97. The kit of clause 91, wherein the adduct-forming agent is a diadduct-forming crosslinking agent.
   98. The kit of clause 97, wherein the diadduct-forming crosslinking agent is a furanocoumarin crosslinking agent.
   99. The kit of clause 98, wherein the furanocoumarin crosslinking agent is a linear furanocoumarin crosslinking agent.
   100. The kit of clause 99, wherein the linear furanocoumarin crosslinking agent is a psoralen crosslinking agent.
   101. The kit of clause 100, wherein the psoralen crosslinking agent is 4,5′,8-trimethylpsoralen.
   102. The kit of any one of clauses 97 to 101, further comprising a crosslink-reversing reagent.
   103. The kit of clause 102, wherein the crosslink-reversing reagent is an alkali crosslink-reversing reagent.
   104. The kit of any one of clauses 97 to 103, further comprising a hairpin adapter molecule, and wherein the instructions comprise instructions for linking the ends of crosslinked genomic DNA using the hairpin adapter molecule.
   105. The kit of any one of clauses 91 to 104, further comprising a nanopore sequencing adapter molecule to facilitate detection of the locations of the adducts in the genomic DNA using a nanopore.

The following examples are offered by way of illustration and not by way of limitation.

### EXPERIMENTAL

### Example 1—Nanopore-Based Identification of Adducts in Yeast Genomic DNA

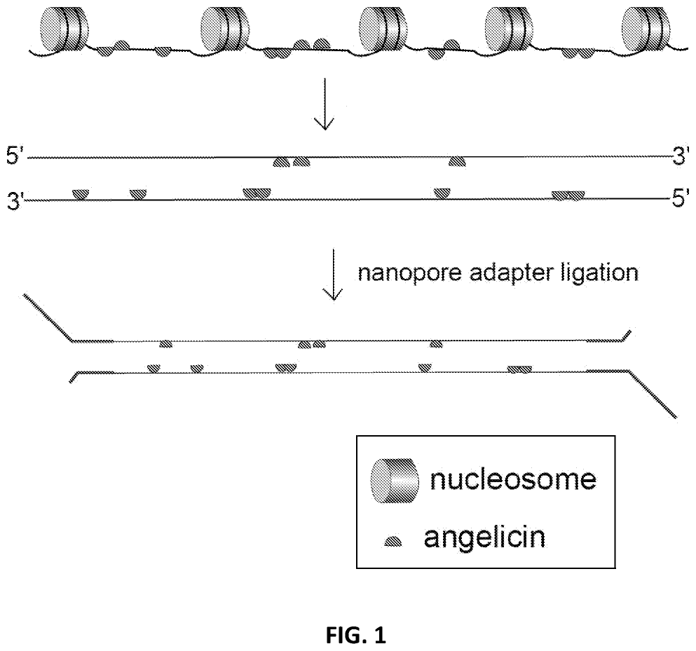

In this example, yeast cells were treated with angelicin and irradiated with UV light to form angelicin monoadducts in unbound/accessible regions of the yeast genomic DNA. Nanopore sequencing adapters were then added to the monoadduct-containing yeast genomic DNA. This approach is schematically illustrated in FIG. 1. The monoadduct-containing yeast genomic DNA was then subjected to nanopore-based analysis using a nanopore-based sequencing device (Oxford Nanopore Technologies). Analysis of a region of the yeast gene RHO4 revealed the detection of angelicin monoadducts. Accordingly, results obtained from the nanopore-based sequencing device confirmed that the locations of angelicin monoadducts in genomic DNA can indeed be detected.

### Example 2—Library Preparation for Nanopore-Based Determination of Nucleosome Positions in Genomic DNA

An example approach for preparing a nucleic acid library for subsequent nanopore-based determination of nucleosome positions in genomic DNA is described herein and illustrated in FIG. 3. In this particular example, cells of interest are treated with 4,5′,8-trimethylpsoralen and, after irradiation with UV light, linker DNA (DNA not associated with nucleosomes) will crosslink to form diadducts with pyrimidines on adjacent strands of DNA. The nucleosome-associated DNA will not be crosslinked, however, because it is protected from the crosslinking agent.

Next, the DNA is 5′ phosphorylated using T4 Polynucleotide Kinase (PNK) and dA-tailed at 3′ ends. Hairpin adapter molecules are then ligated to the ends. Next, crosslinks are reversed by alkali treatment to give rise to pyrone-side monoadducts. A bracelet will form due to denaturing alkali conditions. Next, the buffer is changed to more neutral pH conditions to allow reannealing of the duplex. Uracil-Specific Excision Reagent (USER) enzyme is used to remove uracil in the hairpin adapter. A final end blunting will result in duplexed DNA containing monoadducts. Finally, nanopore sequencing adapters are added to the blunt-ended duplexed DNA containing monoadducts. The sequencing adapted duplexed DNA containing monoadducts is then ready for inputting into a nanopore-based sequencing platform of interest, such as a nanopore-based sequencing platform provided by Oxford Nanopore Technologies, e.g., a MinION™, GridIONx5™, PromethION™, or SmidgION™ nanopore-based sequencing platform.

### REFERENCES

1. Buenrostro J D, Wu B, Chang H Y, Greenleaf W J. ATAC-seq: A Method for Assaying Chromatin Accessibility Genome-Wide. Curr Protoc Mol Biol. 2015; 109: 21.29.1-9.
2. Campbell J D, Alexandrov A, Kim J, Wala J, Berger A H, Pedamallu C S, et al. Distinct patterns of somatic genome alterations in lung adenocarcinomas and squamous cell carcinomas. Nat Genet. 2016; 48: 607-616.
3. Andersson R, Enroth S, Rada-Iglesias A, Wadelius C, Komorowski J. Nucleosomes are well positioned in exons and carry characteristic histone modifications. Genome Res. 2009; 19: 1732-1741.
4. Tolstorukov M Y, Sansam C G, Lu P, Koellhoffer E C, Helming K C, Alver B H, et al. Swi/Snf chromatin remodeling/tumor suppressor complex establishes nucleosome occupancy at target promoters. Proceedings of the National Academy of Sciences. 2013; 110: 10165-10170.
5. Brooks A N, Yang L, Duff M O, Hansen K D, Park J W, Dudoit S, et al. Conservation of an RNA regulatory map between Drosophila and mammals. Genome Res. 2011; 21: 193-202.
6. Brooks A N, Choi P S, de Waal L, Sharifnia T, lmielinski M, Saksena G, et al. A pan-cancer analysis of transcriptome changes associated with somatic mutations in U2AF1 reveals commonly altered splicing events. PLoS One. 2014; 9: e87361.
7. Wang L, Brooks A N, Fan J, Wan Y, Gambe R, Li S, et al. Transcriptomic Characterization of SF3B1 Mutation Reveals Its Pleiotropic Effects in Chronic

Lymphocytic Leukemia. Cancer Cell. 2016; 30: 750-763.
8. Brown C R, Mao C, Falkovskaia E, Jurica M S, Boeger H. Linking stochastic fluctuations in chromatin structure and gene expression. PLoS Biol. 2013; 11: e1001621.
9. Brown C R, Boeger H. Nucleosomal promoter variation generates gene expression noise. Proc Natl Acad Sci USA. 2014; 111: 17893-17898.
10. Komura J, Ikehata H, Hosoi Y, Riggs A D, Ono T. Mapping psoralen cross-links at the nucleotide level in mammalian cells: suppression of cross-linking at transcription factor- or nucleosome-binding sites. Biochemistry. 2001; 40: 4096-4105.
11. Jain M, Koren S, Quick J, Rand A C, Sasani T A, Tyson J R, et al. Nanopore sequencing and assembly of a human genome with ultra-long reads [Internet]. bioRxiv. 2017. p. 128835. doi:10.1101/128835
12. Feng, Yanxiao, Yuechuan Zhang, Cuifeng Ying, Deqiang Wang, and Chunlei Du. 2015. “Nanopore-Based Fourth-Generation DNA Sequencing Technology.” Genomics, Proteomics & Bioinformatics 13 (1): 4-16.
13. Rand A C, Jain M, Eizenga J M, Musselman-Brown A, Olsen H E, Akeson M, et al. Mapping DNA methylation with high-throughput nanopore sequencing. Nat Methods. 2017; 14: 411-413.
14. Simpson J T, Workman R E, Zuzarte P C, David M, Dursi L J, Timp W. Detecting DNA cytosine methylation using nanopore sequencing. Nat Methods. 2017; 14: 407-410.
15. Fatemi M, Pao M M, Jeong S, Gal-Yam E N, Egger G, Weisenberger D J, et al. Footprinting of mammalian promoters: use of a CpG DNA methyltransferase revealing nucleosome positions at a single molecule level. Nucleic Acids Res. 2005; 33: e176.
16. Carter J M, Hussain S. Robust long-read native DNA sequencing using the ONT CsgG Nanopore system. Wellcome Open Res. 2017 Apr. 6; 2:23.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A method for determining nucleosome positions in genomic DNA, comprising:
forming adducts in genomic DNA that mark the locations of linker genomic DNA in the genomic DNA; and
detecting the locations of the adducts in the genomic DNA using a nanopore,
wherein nucleosome positions in the genomic DNA are determined based on the absence of adducts.

2. The method according to claim 1, wherein forming adducts in the genomic DNA comprises forming monoadducts in the genomic DNA.

3. The method according to claim 2, wherein forming monoadducts in the genomic DNA comprises combining the genomic DNA with a monoadduct-forming agent, and treating the genomic DNA and the monoadduct-forming agent to form the monoadducts in the genomic DNA.

4. The method according to claim 3, wherein treating the genomic DNA and the monoadduct-forming agent comprises exposing the genomic DNA and the monoadduct-forming agent to ultraviolet light.

5. The method according to claim 4, wherein the monoadduct-forming agent is a DNA intercalating agent.

6. The method according to claim 5, wherein the DNA intercalating agent is a furanocoumarin compound.

7. The method according to claim 6, wherein the furanocoumarin compound is an angular furanocoumarin compound.

8. The method according to claim 7, wherein the angular furanocoumarin compound is angelicin.

9. The method according to claim 2, wherein forming monoadducts in the genomic DNA comprises:
crosslinking the genomic DNA with a diadduct-forming crosslinking agent such that linker genomic DNA is crosslinked and nucleosome-associated genomic DNA is not crosslinked; and
reversing the crosslinks to form monoadducts from the diadducts.

10. The method according to claim 9, wherein the diadduct-forming crosslinking agent is a furanocoumarin crosslinking agent.

11. The method according to claim 10, wherein the furanocoumarin crosslinking agent is a linear furanocoumarin crosslinking agent.

12. The method according to claim 11, wherein the linear furanocoumarin crosslinking agent is a psoralen crosslinking agent.

13. The method according to claim 12, wherein the psoralen crosslinking agent is 4,5′,8-trimethylpsoralen.

14. The method according to any one of claims 9 to 13, wherein the genomic DNA is present in a cell during the crosslinking.

15. The method according to any one of claims 9 to 14, further comprising, subsequent to the crosslinking and prior to reversing the crosslinks, linking the ends of the genomic DNA.

16. The method according to claim 15, wherein linking the ends of the genomic DNA comprises ligating a hairpin adapter molecule to the ends of the genomic DNA.

17. The method according to claim 16, wherein subsequent to reversing the crosslinks, cutting the hairpin adapter.

18. The method according to claim 17, wherein the hairpin adapter comprises a uracil, and wherein cutting the hairpin adapter comprises excising the uracil from the hairpin adapter.

19. The method according to any one of claims 9 to 18, wherein reversing the crosslinks comprises contacting the cross-linked linker genomic DNA with an alkaline solution.

20. The method according to any one of claims 9 to 19, wherein subsequent to reversing the crosslinks, treating the ends of the genomic DNA to produce blunt ends.

21. The method according to any one of claims 1 to 20, wherein prior to detecting the locations of the adducts in the genomic DNA using a nanopore, adding one or more nanopore sequencing adapters to one or more ends of the genomic DNA.

22. The method according to any one of claims 1 to 21, wherein detecting the locations of the adducts in the genomic DNA using a nanopore comprises:
applying a potential difference across the nanopore;
exposing one or both strands of the genomic DNA to the nanopore in a sequential manner; and
detecting electrical signals from the nanopore corresponding to the adducts in the genomic DNA.

23. The method according to claim 22, wherein a processive enzyme controls the rate of exposure of one or both strands of the genomic DNA to the nanopore in the sequential manner.

24. The method according to claim 22 or claim 23, wherein exposing one or both strands of the genomic DNA to the nanopore in a sequential manner comprises translocating at least a portion of one or both strands of the genomic DNA through the nanopore.

25. The method according to any one of claims 1 to 24, wherein the locations of adducts are detected in a contiguous stretch of genomic DNA of 5 kilobases (kb) or greater.

26. The method according to claim 25, wherein the locations of adducts are detected in a contiguous stretch of genomic DNA of 10 kb or greater.

27. The method according to any one of claims 1 to 26, further comprising sequencing at least a portion of the genomic DNA using the nanopore.

28. The method according to any one of claims 1 to 27, wherein nucleosome positions in the genomic DNA are determined using a computational method.

29. The method according to any one of claims 1 to 28, further comprising assessing mRNA processing in the source of the genomic DNA based on the nucleosome positions in the genomic DNA.

30. The method according to any one of claims 1 to 29, wherein the genomic DNA is yeast genomic DNA.

31. The method according to any one of claims 1 to 29, wherein the genomic DNA is mammalian genomic DNA.

32. The method according to claim 31, wherein the genomic DNA is tumor genomic DNA.

33. A system, comprising:
a device, comprising:
a substrate having a nanopore therein, the substrate separating a first fluid chamber from a second fluid chamber;
a power source electrically coupled to electrodes, wherein the power source and electrodes are adapted to apply a potential difference between the first fluid chamber and the second fluid chamber; and
instructions that cause the system to:
apply a potential difference between the first fluid chamber and the second fluid chamber such that genomic DNA in the first fluid chamber is drawn toward the second fluid chamber and exposed to the nanopore in a sequential manner, wherein the genomic DNA comprises adducts that mark the locations of linker genomic DNA in the genomic DNA;
detect electrical signals from the nanopore corresponding to the adducts; and
record the locations of the adducts in the genomic DNA.

34. The system of claim 33, wherein the instructions further cause the system to sequence at least a portion of the genomic DNA using the nanopore.

35. The system of claim 33 or claim 34, wherein the instructions cause the system to detect electrical signals from the nanopore corresponding to the adducts in a contiguous stretch of the genomic DNA of 5 kilobases (kb) or greater.

36. The system of claim 35, wherein the instructions cause the system to detect electrical signals from the nanopore corresponding to the adducts in a contiguous stretch of the genomic DNA of 10 kb or greater.

37. The system of any one of claims 33 to 36, wherein exposing the genomic DNA to the nanopore in a sequential manner comprises translocating at least a portion of the genomic DNA through the nanopore.

38. The system of any one of claims 33 to 37, comprising a processive enzyme that controls the rate of exposure of the genomic DNA to the nanopore in the sequential manner.

39. The system of any one of claims 33 to 38, wherein the instructions cause the system to determine nucleosome positions in the genomic DNA based on the absence of adducts.

40. The system of claim 39, wherein the instructions cause the system to assess mRNA processing in the source of the genomic DNA based on the nucleosome positions in the genomic DNA.

41. A kit, comprising:
an adduct-forming agent that forms adducts in genomic DNA that mark the locations of linker genomic DNA in the genomic DNA; and
instructions for using the adduct-forming agent in a method for determining nucleosome positions in genomic DNA by detecting the locations of adducts in the genomic DNA using a nanopore.

42. The kit of claim 41, wherein the adduct-forming agent is a monoadduct-forming agent.

43. The kit of claim 42, wherein the monoadduct-forming agent is a DNA intercalating agent.

44. The kit of claim 43, wherein the DNA intercalating agent is a furanocoumarin compound.

45. The kit of claim 44, wherein the furanocoumarin compound is an angular furanocoumarin compound.

46. The kit of claim 45, wherein the angular furanocoumarin compound is angelicin.

47. The kit of claim 41, wherein the adduct-forming agent is a diadduct-forming crosslinking agent.

48. The kit of claim 47, wherein the diadduct-forming crosslinking agent is a furanocoumarin crosslinking agent.

49. The kit of claim 48, wherein the furanocoumarin crosslinking agent is a linear furanocoumarin crosslinking agent.

50. The kit of claim 49, wherein the linear furanocoumarin crosslinking agent is a psoralen crosslinking agent.

51. The kit of claim 50, wherein the psoralen crosslinking agent is 4,5′,8-trimethylpsoralen.

52. The kit of any one of claims 47 to 51, further comprising a crosslink-reversing reagent.

53. The kit of claim 52, wherein the crosslink-reversing reagent is an alkali crosslink-reversing reagent.

54. The kit of any one of claims 47 to 53, further comprising a hairpin adapter molecule, and wherein the instructions comprise instructions for linking the ends of crosslinked genomic DNA using the hairpin adapter molecule.

55. The kit of any one of claims 41 to 54, further comprising a nanopore sequencing adapter molecule to facilitate detection of the locations of the adducts in the genomic DNA using a nanopore.

56. A method for determining bound regions in a double-stranded nucleic acid molecule, comprising:
forming adducts in a double-stranded nucleic acid molecule that mark the locations of unbound regions in the double-stranded nucleic acid; and
detecting the locations of the adducts in the double-stranded nucleic acid using a nanopore,
wherein bound regions of the double-stranded nucleic acid molecule are determined based on the absence of adducts.

57. The method according to claim 56, wherein forming adducts in the double-stranded nucleic acid comprises forming monoadducts in the double-stranded nucleic acid.

58. The method according to claim 57, wherein forming monoadducts in the double-stranded nucleic acid comprises combining the double-stranded nucleic acid with a monoadduct-forming agent, and treating the double-stranded nucleic acid and the monoadduct-forming agent to form the monoadducts in the double-stranded nucleic acid.

59. The method according to claim 58, wherein treating the double-stranded nucleic acid and the monoadduct-forming agent comprises exposing the double-stranded nucleic acid and the monoadduct-forming agent to ultraviolet light.

60. The method according to claim 58 or 59, wherein the monoadduct-forming agent is a DNA intercalating agent.

61. The method according to claim 60, wherein the DNA intercalating agent is a furanocoumarin compound.

62. The method according to claim 61, wherein the furanocoumarin compound is an angular furanocoumarin compound.

63. The method according to claim 62, wherein the angular furanocoumarin compound is angelicin.

64. The method according to claim 57, wherein forming monoadducts in the double-stranded nucleic acid comprises:
crosslinking the double-stranded nucleic acid with a diadduct-forming crosslinking agent such that unbound double-stranded nucleic acid is crosslinked and bound double-stranded nucleic acid is not crosslinked; and
reversing the crosslinks to form monoadducts from the diadducts.

65. The method according to claim 64, wherein the diadduct-forming crosslinking agent is a furanocoumarin crosslinking agent.

66. The method according to claim 65, wherein the furanocoumarin crosslinking agent is a linear furanocoumarin crosslinking agent.

67. The method according to claim 66, wherein the linear furanocoumarin crosslinking agent is a psoralen crosslinking agent.

68. The method according to claim 67, wherein the psoralen crosslinking agent is 4,5′,8-trimethylpsoralen.

69. The method according to any one of claims 64 to 68, wherein the double-stranded nucleic acid molecule is present in a cell during the crosslinking.

70. The method according to any one of claims 64 to 69, further comprising, subsequent to the crosslinking and prior to reversing the crosslinks, linking the ends of the double-stranded nucleic acid molecule.

71. The method according to claim 70, wherein linking the ends of the double-stranded nucleic acid molecule comprises ligating a hairpin adapter molecule to the ends of the double-stranded nucleic acid molecule.

72. The method according to claim 71, wherein subsequent to reversing the crosslinks, cutting the hairpin adapter.

73. The method according to claim 72, wherein the hairpin adapter comprises a uracil, and wherein cutting the hairpin adapter comprises excising the uracil from the hairpin adapter.

74. The method according to any one of claims 64 to 73, wherein reversing the crosslinks comprises contacting the cross-linked double-stranded nucleic acid molecule with an alkaline solution.

75. The method according to any one of claims 64 to 74, wherein subsequent to reversing the crosslinks, treating the ends of the double-stranded nucleic acid molecule to produce blunt ends.

76. The method according to any one of claims 56 to 74, wherein prior to detecting the locations of the adducts in the double-stranded nucleic acid molecule using a nanopore, adding one or more nanopore sequencing adapters to one or more ends of the double-stranded nucleic acid molecule.

77. The method according to any one of claims 56 to 76, wherein detecting the locations of the adducts in the double-stranded nucleic acid molecule using a nanopore comprises:
applying a potential difference across the nanopore;
exposing one or both strands of the double-stranded nucleic acid molecule to the nanopore in a sequential manner; and
detecting electrical signals from the nanopore corresponding to the adducts in the double-stranded nucleic acid molecule.

78. The method according to claim 77, wherein a processive enzyme controls the rate of exposure of one or both strands of the double-stranded nucleic acid molecule to the nanopore in the sequential manner.

79. The method according to claim 77 or claim 78, wherein exposing one or both strands of the double-stranded nucleic acid molecule to the nanopore in a sequential manner comprises translocating at least a portion of one or both strands of the double-stranded nucleic acid molecule through the nanopore.

80. The method according to any one of claims 56 to 79, wherein the locations of adducts are detected in a contiguous stretch of a strand of the double-stranded nucleic acid molecule of 1 kilobase (kb) or greater.

81. The method according to claim 80, wherein the locations of adducts are detected in a contiguous stretch of a strand of the double-stranded nucleic acid molecule of 3 kb or greater.

82. The method according to any one of claims 56 to 81, further comprising sequencing at least a portion of one or both strands of the double-stranded nucleic acid molecule using the nanopore.

83. The method according to any one of claims 56 to 82, wherein bound regions in the double-stranded nucleic acid molecule are determined using a computational method.

84. The method according to any one of claims 56 to 83, wherein the double-stranded nucleic acid molecule is double-stranded DNA.

85. The method according to any one of claims 56 to 83, wherein the double-stranded nucleic acid molecule is an RNA strand having secondary structure.

86. A system, comprising:
a device, comprising:
a substrate having a nanopore therein, the substrate separating a first fluid chamber from a second fluid chamber;
a power source electrically coupled to electrodes, wherein the power source and electrodes are adapted to apply a potential difference between the first fluid chamber and the second fluid chamber; and
instructions that cause the system to:
apply a potential difference between the first fluid chamber and the second fluid chamber such that a double-stranded nucleic acid molecule in the first fluid chamber is drawn toward the second fluid chamber and exposed to the nanopore in a sequential manner, wherein the double-stranded nucleic acid molecule comprises adducts that mark the locations of unbound regions of the double-stranded nucleic acid molecule;
detect electrical signals from the nanopore corresponding to the adducts; and
record the locations of the adducts in the double-stranded nucleic acid molecule.

87. The system of claim 86, wherein the instructions further cause the system to sequence at least a portion of the double-stranded nucleic acid molecule using the nanopore.

88. The system of claim 86 or claim 87, wherein exposing the double-stranded nucleic acid molecule to the nanopore in a sequential manner comprises translocating at least a portion of the double-stranded nucleic acid molecule through the nanopore.

89. The system of any one of claims 86 to 88, comprising a processive enzyme that controls the rate of exposure of the double-stranded nucleic acid molecule to the nanopore in the sequential manner.

90. The system of any one of claims 86 to 89, wherein the instructions cause the system to determine bound regions in the double-stranded nucleic acid molecule based on the absence of adducts.

91. A kit, comprising:
an adduct-forming agent that forms adducts in a double-stranded nucleic acid molecule that mark the locations of unbound regions of the double-stranded nucleic acid molecule; and
instructions for using the adduct-forming agent in a method for determining bound regions in a double-stranded nucleic acid molecule by detecting the locations of adducts in the double-stranded nucleic acid molecule using a nanopore.

92. The kit of claim 91, wherein the adduct-forming agent is a monoadduct-forming agent.

93. The kit of claim 92, wherein the monoadduct-forming agent is a DNA intercalating agent.

94. The kit of claim 93, wherein the DNA intercalating agent is a furanocoumarin compound.

95. The kit of claim 94, wherein the furanocoumarin compound is an angular furanocoumarin compound.

96. The kit of claim 95, wherein the angular furanocoumarin compound is angelicin.

97. The kit of claim 91, wherein the adduct-forming agent is a diadduct-forming crosslinking agent.

98. The kit of claim 97, wherein the diadduct-forming crosslinking agent is a furanocoumarin crosslinking agent.

99. The kit of claim 98, wherein the furanocoumarin crosslinking agent is a linear furanocoumarin crosslinking agent.

100. The kit of claim 99, wherein the linear furanocoumarin crosslinking agent is a psoralen crosslinking agent.

101. The kit of claim 100, wherein the psoralen crosslinking agent is 4,5′,8-trimethylpsoralen.

102. The kit of any one of claims 97 to 101, further comprising a crosslink-reversing reagent.

103. The kit of claim 102, wherein the crosslink-reversing reagent is an alkali crosslink-reversing reagent.

104. The kit of any one of claims 97 to 103, further comprising a hairpin adapter molecule, and wherein the instructions comprise instructions for linking the ends of crosslinked genomic DNA using the hairpin adapter molecule.

105. The kit of any one of claims 91 to 104, further comprising a nanopore sequencing adapter molecule to facilitate detection of the locations of the adducts in the genomic DNA using a nanopore.
